(19) United States
(12) Patent Application Publication
(10) Pub. No.: US 2015/0150572 A1
(43) Pub. Date: Jun. 4, 2015

(54) Dual Balloon Biliary Stone Extraction Device

(71) Applicant: THE JOHNS HOPKINS UNIVERSITY, Baltimore, MD (US)

(72) Inventors: Vivek Kumbhari, Baltimore, MD (US); Mouen Khashab, Towson, MD (US); Florin Selaru, Catonsville, MD (US)

(21) Appl. No.: 14/560,078

(22) Filed: Dec. 4, 2014

Related U.S. Application Data
(60) Provisional application No. 61/911,608, filed on Dec. 4, 2013.

(51) Int. Cl. A61B 17/22; A61M 25/10; A61B 17/12

(52) U.S. Cl. CPC A61B 17/22032; A61B 17/12136; A61M 25/1011; A61M 2210/1042; A61B 2017/22038; A61M 2025/105; A61B 2017/00818

(57) ABSTRACT

The present invention is directed to a method and apparatus for selective guide wire cannulation of a bile duct, removal of biliary calculi, and delivering hydrodynamic gene delivery are also provided. There is provided a method for straightening the terminal common bile duct to allow biliary calculi to be removed, assisting in creation of a gastoejunostomy, and selective guidewire cannulation. A method for isolating a segment of biliary epithelium for hydrodynamic gene delivery is also provided, in which a segment of the bile duct is isolated and plasmids (oncogenic or therapeutic) are injected under pressure to allow entry into cholangiocytes. There is also provided a method for isolating the cystic duct or left/right intrahepatic ducts to allow the insertion of a guide wire and subsequent therapy. A single catheter platform may be used to perform the above procedures.

### CROSS REFERENCE TO RELATED APPLICATIONS

This application claims the benefit of U.S. Provisional Patent Application No. 61/911,608, filed Dec. 4, 2013, which is incorporated by reference herein, in its entirety.

### FIELD OF THE INVENTION

The present invention relates generally to medical devices. More particularly, the present invention relates to a device for removal of biliary stones, selective cannulation of branches of the biliary tree, and delivery of solution to cholangiocytes of the bile duct.

### BACKGROUND OF THE INVENTION

Selective guide wire cannulation of a biliary radicle (cystic duct, left/right intrahepatic duct etc) in patients with pathology located in the cystic duct, gallbladder or branches of the intrahepatic ducts can also be difficult. Such cannulation may be dealt with using the aid of endoscopic retrograde cholangiography (ERC). ERC is standard medical procedure where an endoscope is used to access the common bile duct via the major papilla which is located in the proximal part of the duodenum. There are multiple catheter based accessories that are used to access the common bile duct but the placement of a guide wire is of critical importance to allow these accessories to reach the region of interest. Guide wire placement in the cystic duct and intrahepatic ducts is often a challenge due to the state of current accessories (which only allow the guide wire to exit the catheter at its tip) and their narrow lumen (often 2 mm in diameter). It is conceivable that if a catheter had a port which allowed the guide wire to exit out from the side then this may facilitate selective cannulation of these smaller biliary radicles which would then allow diagnostic and therapeutic interventions to be possible with currently available accessories.

Biliary calculi and debris can become jammed in the distal common bile duct. Approximately 15% of people who develop gallstones will develop choledocholithiasis (stones in the common bile duct). There is a marked variation in the size of stone formation from 2 mm to 20 mm. There is also marked variation in the anatomy from person to person of the common bile duct. One anatomical anomaly is when there is a “kink” or acute angle at the end of the bile duct as it exits into the duodenum. This may occur for a multitude of reasons (peri-ampullary diverticulum, congenital etc). The current standard of care method to remove common bile duct stones is via endoscopic retrograde cholangiography (ERC). Currently, choledocholithiasis are being removed by a stone extraction balloon at endoscopic retrograde cholangiopancratography (ERCP). This is a catheter with a single balloon at the tip which is inflated above the stone and drags the stone out of the bile duct. These stones can get jammed in the end of the bile duct due to a kink or a small biliary opening. Theoretically, if the bile duct is momentarily straightened as the biliary material is being pulled through into the duodenum, the passage of the calculi/debris may be more successful. It is conceivable that a straight catheter with two balloons separated by a short distance and inflated to the size of the bile duct lumen would temporarily straighten the distal bile duct and allow passage of the biliary contents.

Additionally, cholangiocytes are the epithelial cells that line the bile duct. Cholangiocarcinoma (CCA) is a malignant neoplasm derived from cholangiocytes. There is currently no large animal model for CCA. Establishing a preclinical model of CCA that rapidly and reliably recapitulates key pathological features of the advanced human disease, including malignant bile duct obstruction, can provide an important resource for investigating CCA growth and progression. Furthermore, such a patient-like model can potentially be of great value for preclinical testing of endoscopic diagnostic and therapeutic strategies against CCA.

Hydrodynamic gene delivery is one approach to facilitating the conversion of normal cholangiocytes to CCA. This technique involves injection of oncogenic plasmids under high pressure to allow diffusion into cells and integrate into the cellular DNA. This focal area of high pressure can be generated by occluding two ends of a lumen (such as a vein) using an inflatable balloon and injecting the plasmids in between them. Hydrodynamic gene delivery has been used in animal models for the creation of hepatocellular carcinoma and sarcomas. Its delivery method has been limited to the vasculature though there does not appear to be a reason why the same delivery method cannot be used in other anatomical locations such as the bile duct.

Accordingly, there is a need in the art for a catheter configured for removal of bile duct stones, selective cannulation of the biliary tree, and delivery of solution to cholangiocytes of the bile duct.

### SUMMARY OF THE INVENTION

The foregoing needs are met by the present invention which provides a method for providing hydrodynamic delivery of genes to cholangiocytes in a bile duct including inserting a catheter device into the bile duct. The catheter device includes dual balloons positioned in series proximal to a distal end of the catheter device and wherein the dual balloons are independently inflatable. Both balloons are inflated under fluoroscopic vision after injection of a contrast agent into the bile duct. The balloons are inflated to just above the bile duct lumen to occlude it. Then the solution containing plasmids is injected under pressure such that it fills the space between the balloons. After 30 seconds the balloons are then deflated and the catheter can be removed from the bile duct.

In accordance with an aspect of the present invention, the method further includes removing a calculi in the bile duct. The method includes inserting a first balloon of the dual balloons past the calculi in the bile duct and inflating the first balloon and subsequently a second balloon of the dual balloons in order to capture the calculus in between the two balloons. Then the catheter can be withdrawn into the duodenum to allow passage of the calculus. Additionally, the method includes providing hydrodynamic delivery of genes into a biliary epithelium for the creation of a cholangiocarcinoma. Alternately, the method can include providing hydrodynamic delivery of genes into a diseased biliary epithelium as a form of therapy. The method can also include facilitating a selective guide wire cannulation of a cystic duct, and/or facilitating a selective guide wire cannulation of an intrahepatic duct.

In accordance with another aspect of the present invention, the method includes isolating the cystic duct and/or the intrahepatic duct. A cholangiocarcinoma can be created in a large animal as a model. The method further includes straightening the bile duct. The method further includes facilitating a selective cannulation of a bile duct. Additionally, the method includes injecting plasmids under pressure to allow entry into cholangiocytes.

In accordance with still another aspect of the present invention, a method for cannulation of a bile duct includes inserting a catheter device into the bile duct, wherein the catheter device includes dual balloons positioned in series proximal to a distal end of the catheter device and wherein the dual balloons are independently inflatable. The method includes inflating at least one of the balloons to just above a size of the bile duct to create a closed space. The method also includes removing the catheter device from the bile duct.

In accordance with yet another aspect of the present invention, the method includes identifying calculi in the bile duct. The method includes inserting a first balloon of the dual balloons past the calculi in the bile duct and inflating the first balloon and subsequently a second balloon of the dual balloons in order to remove the calculi. The method also includes facilitating a selective guide wire cannulation of a cystic duct and/or facilitating a selective guide wire cannulation of an intrahepatic duct. Additionally, the method includes straightening the bile duct, and facilitating a selective cannulation of a bile duct.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1A illustrates a side view of a double balloon catheter according to an embodiment of the present invention.

FIG. 1B illustrates a cross-sectional view of the catheter, according to an embodiment of the present invention.

FIG. 1C illustrates a cross-sectional view of the catheter, according to another embodiment of the present invention.

FIGS. 2 and 3 illustrate images of a catheter, according to an embodiment of the present invention.

FIG. 4 illustrates an image from an exemplary cholangiogram with the balloons of the present invention inflated in a porcine bile duct.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains, having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention is directed to a method and apparatus for selective guide wire cannulation of a bile duct, removal of biliary calculi, and delivering hydrodynamic gene delivery are also provided. There is provided a method for straightening the terminal common bile duct to allow biliary calculi to be removed, assisting in creation of a gastoejunostomy, and selective guidewire cannulation. A method for isolating a segment of biliary epithelium for hydrodynamic gene delivery is also provided, in which a segment of the bile duct is isolated and plasmids (oncogenic or therapeutic) are injected under pressure to allow entry into cholangiocytes. There is also provided a method for isolating the cystic duct or left/right intrahepatic ducts to allow the insertion of a guide wire and subsequent therapy. A single catheter platform may be used to perform the above procedures.

Firstly, the device is created for the purposes of allowing hydrodynamic gene delivery to cholangiocytes that line the bile duct. The initial purpose of this is to create cholangiocarcinoma (CCA) in a large animal model. Hydrodynamic gene delivery has been used exclusively intravascularly in both small and large animal models. It has however, not been used extravascularly. A model of CCA would reliably recapitulate key pathological features of this advanced human disease. Such a patient-like model can potentially be of great value for preclinical testing of endoscopic diagnostic and therapeutic strategies against CCA. To create this model, a fixed area of the bile duct would have to be sealed off and in between them a certain volume of DNA will have to be injected under pressure so they enter the cholangiocytes. This invention will allow this to occur.

Secondly, the device is created for the purposes of allowing removal of biliary stones from the common bile duct. Approximately 15% of people who develop gallstones will develop choledocholithiasis (stones in the common bile duct). There is a marked variation in the size of stone formation form 2 mm to 20 mm. There is also marked variation in the anatomy from person to person of the common bile duct. One anatomical anomaly is when there is a “kink” or acute angle at the end of the bile duct as it exits into the duodenum. This may occur for a multitude of reasons (peri-ampullary diverticulum, congenital etc). The current standard of care method to remove common bile duct stones is via endoscopic retrograde cholangiopancreatography (ERCP). Currently, choledocholithiasis are being removed by a stone extraction balloon at ERCP. This is a catheter with a single balloon at the tip which is inflated above the stone and drags the stone out of the bile duct. Unfortunately, these stones can get jammed in the end of the bile duct due to the kink or a small biliary opening. This device will have two balloons which by which one can be placed above and another below the stone and then the device can be dragged out the biliary opening. The balloon below the stone will be the first to come out and this will keep the bile duct straight and also keep the bile duct open somewhat to allow the stone to come through.

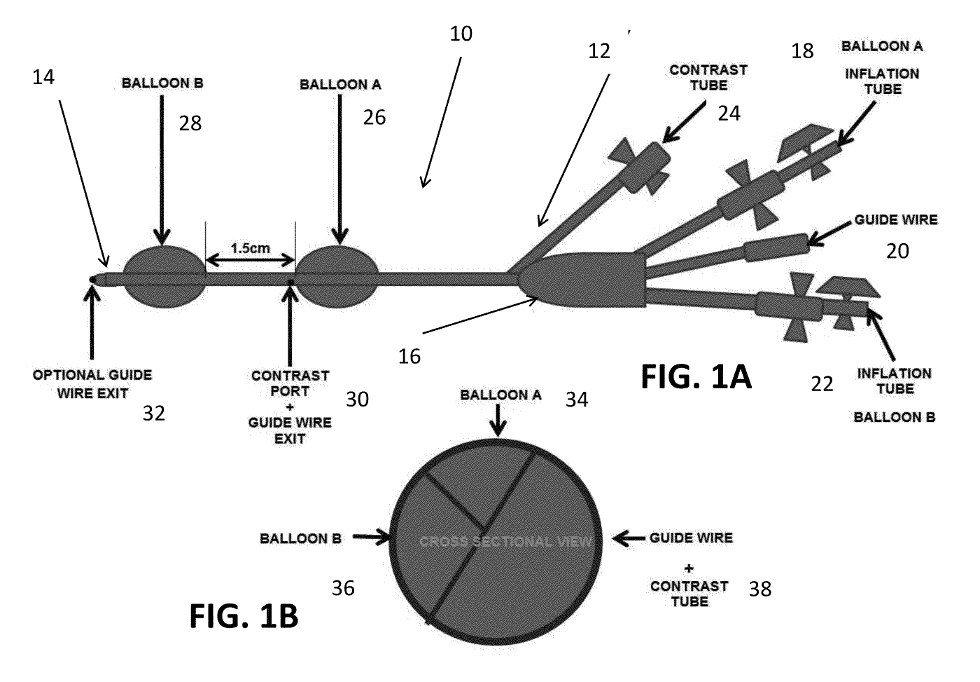

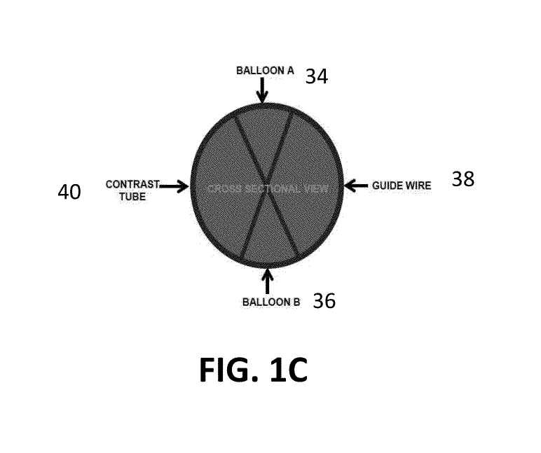

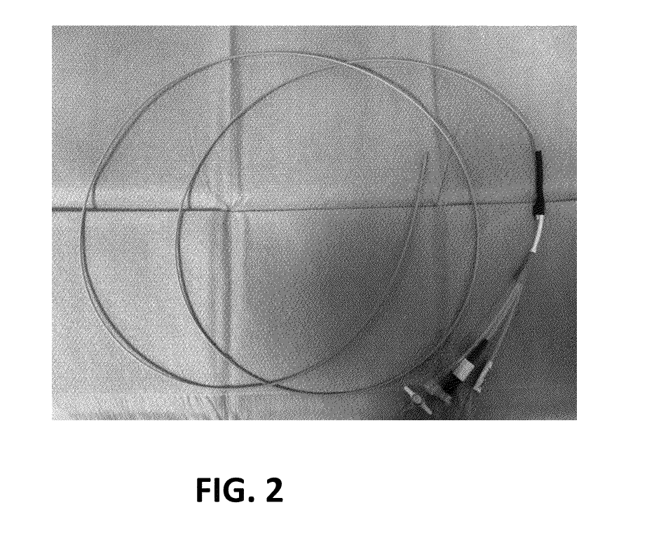

FIG. 1A illustrates a side view of a double balloon catheter according to an embodiment of the present invention. As illustrated in FIG. 1A, the catheter 10 includes a proximal end 12 and a distal end 14. The proximal end 12 of the catheter 10 includes a hub 16 and coupled line ports 18, 20, 22, and 24. While four line ports are shown in FIG. 1A, it would be possible to include more or fewer line ports as needed and known to one of skill in the art. The catheter 10 also includes a first and a second balloon 26, 28 disposed in series just proximal to the distal end 14 of the catheter 10. The first and second balloons 26, 28 can be separated by a length of the catheter 10. Preferably, the distance between the first and second balloons 26, 28 is approximately 15 mm. This distance allows the catheter to be useful for both hydrodynamic delivery and also stone extraction.

Further, as illustrated in FIG. 1A, two of the line ports 18 and 22 are configured as inflation tubes for the first and second balloons 26, 28, respectively. The catheter 10 also includes a guide wire port for inserting and adjusting a guide wire. The guide wire can exit the catheter at the distal end 14 at the guide wire exit 30. A contrast tube 24 is also included in order to allow for delivery of contrast and medication. The contrast can exit through the contrast exit 32. In some embodiments, it is preferable that the guide wire exit 30 and the contrast exit 32 allow for the exit of both a guide wire and contrast or medication.

FIG. 1B illustrates a cross-sectional view of the catheter, according to an embodiment of the present invention. As illustrated in FIG. 1B the catheter includes a first, a second, and a third lumen 34, 36, and 38, respectively. The first and second lumens 34 and 36 are configured to allow for the inflation of the first and second balloons, respectively. The third lumen 38 allows for the insertion of the guidewire and dispensing contrast or medication. FIG. 1C illustrates a cross-sectional view of the catheter, according to another embodiment of the present invention. As illustrated in FIG. 1C the catheter includes a first, a second, a third, and a fourth lumen 34, 36, 38, and 40, respectively. The first and second lumens 34 and 36 are configured to allow for the inflation of the first and second balloons, respectively. The third lumen 38 allows for the insertion of the guidewire, and the fourth lumen 40 for dispensing contrast or medication.

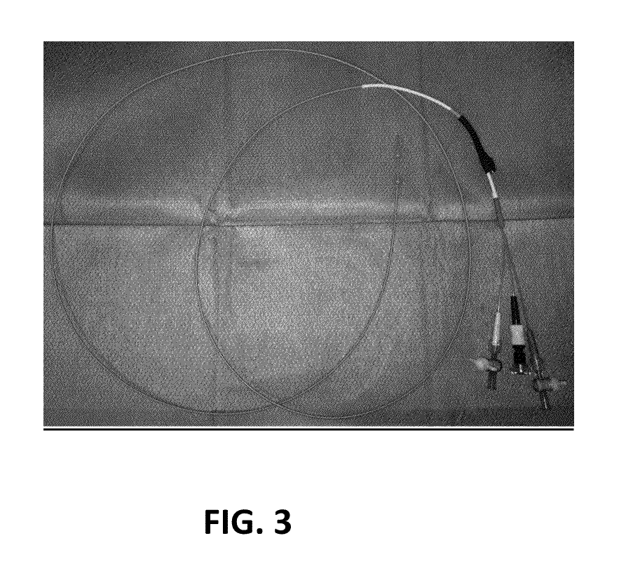

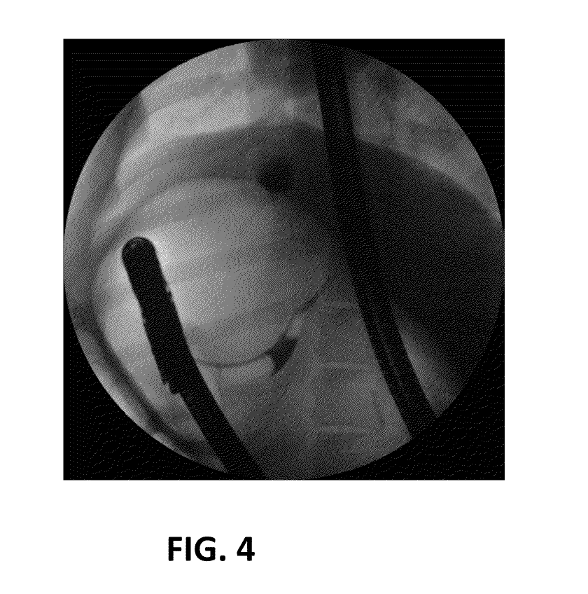

FIGS. 2 and 3 illustrate images of a catheter, according to an embodiment of the present invention. FIG. 2 illustrates the catheter with the first and second balloons in a deflated state, and FIG. 3 illustrates the catheter with the first and second balloons in an inflated state. Additionally, FIG. 4 illustrates an image from an exemplary cholangiogram with a balloon of the present invention inflated in a porcine bile duct. It should be noted that the balloon catheter is preferably approximately 200 cm in length and has an approximate diameter of 6.6 Fr to 7 Fr. The balloons can be inflated to a diameter of approximately 2 cm, and can be formed from a latex or latex substitute material. Additionally, the catheter can include radio-opaque markers at the site of the balloon, for identifying catheter position in the patient or subject.

A product according to the present invention will enhance surgical procedures. For instance, the second balloon allows the stone to be negotiated from the distal biliary structure, kink in the distal bile duct and may obviate the need for a biliary sphicteroplasty. In addition, the present invention may be used in the pancreas to aid in the removal of intraductal stones. The current single balloon device is often unable to remove large stones. Often multiple procedures or accessories such as lithotripsy baskets are necessary to aid in removal. There is a need for this device as biliary and pancreatic stones are the most common indication for ERCP and their incidence is likely to increase over the subsequent years.

It should also be noted that the device is useful for the injection of substances into the left or right intrahepatic duct or cystic duct. If the balloons of the device are placed appropriately then selective injection can occur. This can be used for diagnostic purposes, i.e. contrast injection into a particular system as is preferential in cases of cholangiocarcionoma. Additionally, in the future injection of agents as therapy for malignancy or stones may be useful.

By having two balloons and an injection/guidewire port in between them, the guidewire may be able to be directed into the left or right intrahepatic duct or cystic duct. Selective guidewire cannulation is currently a challenge and the present invention will serve to simplify the procedure.

The two inflated balloons may also promote easier removal of proximally migrated biliary or pancreatic stones. By inflating a balloon above the stent and the other balloon at the level of the stent, the ability to remove the stones increases. There are increasing indications for stenting the biliary and/or pancreatic duct so this is a likely use of the device.

This invention would be useful in removing large stones from the pancreatic duct. The difficulty in removing large stones from the pancreatic duct in patients with chronic calcific pancreatitis is stone to duct size mismatch. When the size of the duct closest to the papilla is smaller than the stone it can be hard for the stone to be dragged through the relatively narrow duct. However, with this device, the second balloon can also be inflated on the side closest to the papilla and this will effectively increase the functional duct size and all the stone to come through with ease. Management of calcific chronic pancreatitis is a major clinical problem with endoscopy only being successful in removing stones in approximately 40% even with extended shock wave lithotripsy. Many patients either go to surgery or simply suffer with chronic pain and vitamin deficiency due to ductal obstruction.

The invention can also be used to direct a substance to be injected towards the stone and due to its chemical properties it simply breaks down. By inflating the two balloons on either side of the stone this substance could be injected into this ‘closed space and this may be sufficient to break down the stone.

Also, the device would be useful in the guiding interventional endoscopic ultrasound procedures such as the creation of a gastrojejunostomy. The method would be to insert the catheter into the jejunum over a guidewire. The balloons would then be inflated and the space between them filled with water. Then under sonographic guidance the water filled area could be identified. This area would then be punctured with a EUS needle and a guidewire deployed. Over this guidewire, a lumen opposing stent could be deployed and hence a gastrojejunostomy created.

### EXEMPLARY EMBODIMENTS

An exemplary protocol for the creation of a common bile duct cholangiocarcinoma for a pig is described below. This illustration is only meant to be an example and other methods of performing the procedure could also be used. The pig will be fasted overnight prior to the procedure but will be allowed free access to water. Prior to the procedure, the pigs will be sedated with a cocktail made of 500 mg Telazol reconstituted with 250 mg of ketamine (100 mg/ml) and 250 mg xylazine (100mg/ml). This cocktail is administered at a dose of 1 ml/50 pounds of body weight. Once sedated, the pig will be intubated and an IV catheter placed in a marginal ear vein. Throughout the procedure, the pig will be administered maintenance doses of 0.9% saline or lactated ringers IV.

The pig will then be moved to the procedure room and attached to the anesthesia machine and ventilator. General anesthesia will be maintained with isoflurane and mechanical ventilation. During the procedure, heart rate, end tidal CO2 and ECG will be monitored. Vitals will be recorded every 15 minutes.

The endoscope will be clean. All participants will be wearing clean gowns and gloves. An intramuscular injection of 600,000 units of penicillin G benzathine and penicillin G procaine based antibiotic and 1 g intravenous (IV) cefazolin may be administered before endoscopy. A duodenoscope (side viewing endoscope) will be passed through the mouth to the duodenum and the biliary opening will be identified. The Endoscopic Retrograde Cholangiography (ERC) and with hydrodynamic gene delivery to the biliary epithelium will begin.

Under X-ray guidance, the bile duct will be accessed with the Dual Balloon device. The device will be inserted such that both balloons are in the common bile duct. A luer lock syringe will be used to aspirate bile so its position is confirm. Further confirmation will be obtained after 4 mls of omnipaque will be in used opacify the biliary tree. The diameter of the common bile duct will then be estimated using the fluoroscopy image. The balloon will be inflated to just above the size of the bile duct so as to create a closed space. Then the bile duct will be injected with 3 mls (or until a resistance to fluid injection is noticed) of a combination of normal saline and DNA (as plasmids from a mouse cell line). After 30 seconds the balloons will be deflated and together with the endoscope will be removed from the pig. The procedure will then be complete.

At the completion of each endoscopy procedure, the anesthetic will be turned off and the pig allowed to recover. All materials to be disposed will be placed in standard biohazard bags and disposed of appropriately in designed containers. Once sternal, the pig will be returned to its home cage and allowed to eat.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A method for cannulation of a duct comprising:
inserting a catheter device into the duct, wherein the catheter device includes dual balloons positioned in series proximal to a distal end of the catheter device and wherein the dual balloons are independently inflatable;
inflating at least one of the balloons to just above a size of the duct to create a closed space; and
removing the catheter device from the duct.

2. The method of claim 1 further comprising identifying a calculi in the duct.

3. The method of claim 2 further comprising inserting a first balloon of the dual balloons past the calculi in the bile duct and inflating the first balloon and subsequently a second balloon of the dual balloons in order to remove the calculi.

4. The method of claim 1 further comprising facilitating a selective guide wire cannulation of a cystic duct.

5. The method of claim 1 further comprising facilitating a selective guide wire cannulation of an intrahepatic duct.

6. The method of claim 1 further comprising straightening the duct.

7. The method of claim 1 further comprising facilitating a selective cannulation of the duct.

8. The method of claim 1 further comprising cannulating a bile duct.

9. The method of claim 1 further comprising cannulating a pancreatic duct.

10. The method of claim 1 further comprising creating a gastroejunostomy.

11. The method of claim 1 further comprising facilitating a selective guidewire cannulation of the duct.

12. A method for providing hydrodynamic delivery of genes to cholangiocytes in a bile duct comprising:
inserting a catheter device into the bile duct, wherein the catheter device includes dual balloons positioned in series proximal to a distal end of the catheter device and wherein the dual balloons are independently inflatable;
inflating at least one of the balloons to just above a size of the bile duct to create a closed space;
injecting the bile duct with the genetic material; and
removing the catheter device from the bile duct.

13. The method of claim 12 further comprising providing hydrodynamic delivery of genes into a biliary epithelium for the creation of a cholangiocarcinoma.

14. The method of claim 13 further comprising providing hydrodynamic delivery of genes into a diseased biliary epithelium as a form of therapy.

15. The method of claim 12 further comprising facilitating a selective guide wire cannulation of a cystic duct.

16. The method of claim 15 further comprising isolating the cystic duct.

17. The method of claim 12 further comprising facilitating a selective guide wire cannulation of an intrahepatic duct.

18. The method of claim 17 further comprising isolating the intrahepatic duct.

19. The method of claim 12 further comprising creating a cholangiocarcinoma in a large animal as a model.

20. The method of claim 12 further comprising injecting plasmids under pressure to allow entry into cholangiocytes.
